(19) United States
(12) Patent Application Publication
(10) Pub. No.: US 2009/0099307 A1
(43) Pub. Date: Apr. 16, 2009

(54) Inverse solid phase peptide synthesis with additional capping step

(75) Inventor: Ram Prakash Sharma, Southampton (GB)

Correspondence Address: STEPTOE & JOHNSON LLP, 1330 CONNECTICUT AVENUE, N.W., WASHINGTON, DC, 20036, US

(73) Assignee: ACTIVOTEC SPP LIMITED, Cambridgeshire (GB)

(21) Appl. No.: 11/886,184

(22) PCT Filed: Mar. 13, 2006
(86) PCT No.: PCT/GB2006/000872

§ 371 Date: May 23, 2008

(30) Foreign Application Priority Data
Mar. 14, 2005 (GB) 0505221.2

(51) Int. Cl. C07K 1/04

(52) U.S. Cl. 525/54.11

(57) ABSTRACT

A process for preparing a peptide by solid phase synthesis in the N to C direction comprising a capping step after an amide bond formation step to prevent or reduce deletion artefacts.

The present invention relates to a process for the preparation of peptides and proteins by solid phase synthesis and to peptides and proteins obtainable by such a process.

Peptides and proteins are composed of the amino acids. There are about 20 different amino acids commonly available in nature, and they are linked together in long chains to form peptides. Biologically active peptides, consisting of between 2 and 50 amino acids, span a wide range of functions in nature: hormones, chemokines, neurotransmitters, cytokines and immunological agents. They have also been shown to be effective as prophylactic and therapeutic vaccines as well as enzyme inhibitors.

Protein therapeutics has emerged as one of the most promising segments of the pharmaceutical market since the introduction of recombinant insulin in 1982. To produce these important drugs commercially, companies have focused to date on biological approaches such as recombinant-DNA expression methods (microbial fermentation and mammalian cell culture) and native protein isolation. However numerous problems are associated with these methods:
a) limited supply of product is possible
b) viral contamination risk
c) product heterogeneity
d) inability to produce some proteins e.g. those that are toxic to the cell
e) non-human post-translational modifications, i.e. incorrect glycosylation or folding
f) time-consuming
g) structural modifications are limited to the 20 naturally occurring amino acids

Chemical protein synthesis provides a rapid and efficient route for the production of homogenous proteins containing up to 250 amino acids that are free of biological contaminants. In this field the development of solid-phase peptide synthesis (SPPS) by Merrifield in 1963 merited the award of Nobel prize in 1984 (Merrifield, R. B. (1963) J. Amer. Chem. Soc., 85,2149-2154). This method is still widely used. However this method originally only allowed efficient production of small peptides, for example up to about 10 kDa, such as hormones and cytokines. Another significant limitation of this method is incomplete synthesis and side reactions.

The present inventors have made advances towards reversing the conventional C-to-N direction of synthesis and a new approach to synthesising peptides to allow the preparation on the solid-phase of peptide analogues possessing C-terminal modifications (such as esters, thioesters, alcohols, aldehydes and others), peptides possessing peptide bond modifications (such as reduced peptide bonds, urea, and isosteres) as well as to facilitate fragment coupling on the solid-phase. This N to C method is disclosed in Sharma, R. P., Jones, D. A., Corina, D. L. and Akhtar, M. (1994) in Peptides: Chemistry, Structure and Biology, Proceedings of the Thirteenth American Peptide Symposium (Hodges, R. S. and Smith, J. A., eds.), pp. 127-129, ESCOM, Leiden, Jones, D. A. (1993) PhD Thesis, University of Southampton, and WO93/65065.

In the past, the lack of a suitable protection for the carboxyl group which could be removed under mild conditions had hindered progress in this area. Earlier attempts at the solid-phase peptide synthesis in the N-to-C direction (Letsinger, R. L. and Kornet, M. J. (1963) J. Amer. Chem. Soc., 85, 3045-3046, Letsinger, R. L., Kornet, M. J., Mahadevan, V. and Jerina, D. M. (1964) J. Amer. Chem. Soc., 86, 5163-, Felix, A. M. and Merrifield, R. B. (1970) J. Amer. Chem. Soc., 92, 1385-1391) were hindered by the use of amino acid esters that were effectively too stable. The conditions required for the removal of the ester protection before commencing the next addition cycle as a consequence were very harsh. In order to improve this situation the present inventors employ more suitable amino acid ester building blocks and have developed the use of trialkoxy silyl (tBos) esters of amino acids for use in solid-phase peptide synthesis in the N-to-C direction. These derivatives can be readily prepared, are inexpensive, stable throughout the coupling reaction and the protecting group can be selectively removed in good yield under mild acid conditions before commencing the next cycle.

Although the N-to-C solid phase peptide synthesis methodology employing trialkoxy silyl (tBos) esters of amino acids represents a great improvement on previously known methodologies based on trialkylsilyl esters, it does suffer from certain drawbacks. Although the individual coupling reactions of (tBos) esters of amino acids to the free acid of the resin-bound peptide chain proceed in good yield, inevitably some proportion of free acid remains unreacted. These uncoupled free acids are able to react in subsequent amino acid couplings. The result is that the end product is contaminated with impurities differing from the desired product by the omission of only one or two amino acid residues. These are known as “deletion artefacts” and it is extremely difficult to free the final product from these contaminants.

The present invention provides novel processes for the synthesis of peptides and proteins in the N-to-C direction without the limitations and disadvantages of the prior art.

According to a first aspect, the present invention relates to a process for the preparation of a solid support-bound peptide of general formula (I)

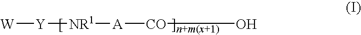

which comprises the following steps:

(a) reacting a solid-support bound amino acid derivative (II) with a carboxyl group activating agent to form an activated solid support-bound compound of formula (III):

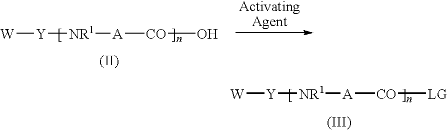

(b) reacting the activated solid support-bound compound of formula (III) with a second carboxy-protected amino acid or peptide derivative of formula (IV) to form a solid support-bound peptide chain extended compound of formula (V);

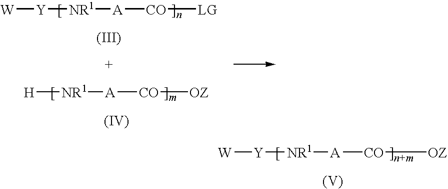

(c) treating the solid support-bound peptide chain extended compound of formula (V) with a capping agent;

(d) removing the protecting group Z to produce a solid support-bound peptide derivative of formula (VI);

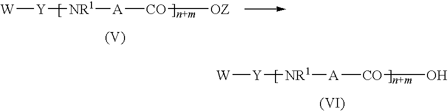

(e) repeating steps (a), (b), (c) and (d) x times to form the desired compound of general formula (I);

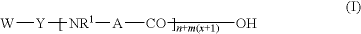

(f) optionally, cleaving the chain-extended compound at the N—Y bond to form a free peptide of formula (VII);

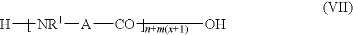

wherein
n is a positive integer
m is a positive integer
x is 0 or a positive integer
Z is a carboxy protecting group
W is a solid support
Y is a linker group or a chemical bond
LG is a leaving group
R¹ is hydrogen or a substituent
and for each A, which may be the same or different,
   i) A represents the amino acid residue; or
   ii) A, taken together with R¹ and N, forms a heterocycle (for example in the case of proline);

The amino acids can be natural, unnatural or modified. They can be added together singly or as small peptides or modified peptides. The residues A of the amino acids may incorporate protected functional groups.

By use of the term “solid support” we mean the support onto which the amino acids are linked, optionally through a linker. The supports include solid and soluble solid materials or matrixes, and resins.

In a preferred aspect, the completed peptide is finally released from the solid support.

Preferred solid supports W for N—C synthesis are derivatised Merrifield resins, that is resins based on chloromethylstyrene/divinylbenzene copolymers. Particularly useful resins are PEG-PS e.g. Tentagel (obtained from Novabiochem) which have increased tolerance to aqueous media.

By “leaving group” it is meant any chemical moiety which is capable of detachment from the acyl group of the amino acid with the concomitant formation of a new amide bond. Many suitable leaving groups will be known to those skilled in the art. Examples of particularly suitable leaving groups include:

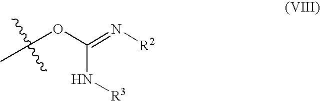

i) derivatives of carbodiimides of formula (VIII)

wherein R² and R³ are independently C₁₋₁₀ hydrocarbyl groups, preferably cyclohexyl;
ii) derivatives of pentafluorophenol, hydroxybenzatriazole, hydroxysuccinimide, 1-hydroxy-7-azabenzotriazole, carbonyldiimidazole, 3-hydroxy-3,4-dihydro-4-oxo-1,2,3-benzotriazine, N-ethyl-5-phenylisoxazolium-3′-sulphonate;
iii) halides, particularly fluoride.

A particularly preferred leaving group is oxybenzotriazole (—OBt).

By “activating agent” it is meant any reagent or combination of reagents that is capable of converting the free carboxylic acid group of an amino acid or peptide fragment to an activated form, in which the acyl carbon bears a leaving group LG as defined above. Many activating agents have proved useful in this capacity, and the skilled man will have little difficulty in selecting an appropriate one.

Preferred activating agents are selected from:
i) carbodiimides, including 1,3-dicyclohexylcarbodiimide (DCC); 1-ethyl-3-(3′-dimethylaminopropyl)carbodiimide hydrochloride, (EDCl), optionally with base;
ii) aminium/uronium based reagents, including 1-benzotriazol-1-yloxy-bis(pyrrolidino)uronium hexafluorophosphate, 5-(1H-benzotriazol-1-yloxy)-3,4-dihydro-1-methyl 2H-pyrrolium hexachloroanitimonate, benzotriazol-1-yloxy-N,N-dimethylmethaniminium hexachloroantimonate, O-(7-azabenzotriazol-1-yl)-1,1,3,3-tetramethyluronium hexafluorophosphate, O-(7-azabenzotriazol-1-yl)-1,1,3,3-bis(tetramethylene)uronium hexafluorophosphate, O-(benzotriazol-1-yl)-1,1,3,3-tetramethyluronium hexafluorophosphate, O-(7-azabenzotriazol-1-yl)-1,1,3,3-bis(pentamethylene)uronium tetrafluoroborate, 2-(3,4-dihydro-4-oxo-1,2,3-benzotriazin-3-yl)-1,1,3,3-tetramethyluronium tetrafluoroborate, 2-(5-norbornene-2,3-dicarboximido)-1,1,3,3-tetramethyluronium tetrafluoroborate, 2-(2-oxo-1(2H)-pyridyl-1,1,3,3-tetramethyluronium tetrafluoroborate, 2-succinimido-1,1,3,3-tetramethyluronium tetrafluoroborate, optionally in combination with base;
iii) phosphonium based reagents including O-(7-azabenzotriazol-1-yl)-tris(dimethylamino)phosphonium hexafluorophosphate benzotriazol-1-yl diethyl phosphate1-benzotriazolyoxytris(dimethylamino)phosphonium hexafluorophosphate (Castro's Reagent), 7-azobenzotriazolyoxytris(pyrrolidino)phosphonium hexafluorophosphate, 1-benzotriazolyoxytris(pyrrolidino)phosphonium hexafluorophosphate, optionally in combination with base;
iv) other peptide coupling reagents including 2-bromo-3-ethyl-4-methyl thiazolium tetrafluoroborate, bis(2-oxo-3-oxazolidinyl)phosphinic chloride, bromotris(dimethylamino)phosphonium hexafluorophosphate, bis(tetramethylenefluoroformamidinium) hexafluorophosphate, 2-chloro-1,3-dimethylimidazolidinium hexafluorophosphate, 3-(diethoxyphosphoryloxy)-1,2,3-benzotriazin-4(3H)-one, diphenylphosphinic chloride, 2-ethoxy-1-ethoxycarbonyl-1,2-dihydroquinoline, pentafluorophenyl diphenylphosphinate, S-(1-oxido-2-pyridinyl)-1,1,3,3-tetramethylthiouronium hexafluorophosphate, bromotris(pyrrolydino)phophonium hexafluorophosphate, chlorotris(pyrrolydino)phophonium hexafluorophosphate, tetramethylfluoroformamidinium hexafluorophosphate, S-(1-oxido-2-pyridinyl)-1,1,3,3-tetramethylthiouronium tetrafluoroborate, optionally in combination with base.

Optionally, the activating agent includes at least one activating additive. Preferred activating additives include pentafluorophenol, hydroxybenzatriazole, hydroxysuccinimide, 1-hydroxy-7-azabenzotriazole, carbonyldiimidazole, 3-hydroxy-3,4-dihydro-4-oxo-1,2,3-benzotriazine or N-ethyl-5-phenylisoxazolium-3′-sulphonate.

A preferred activating agent is a combination of 1-benzotriazolyoxytris(dimethylamino)phosphonium hexafluorophosphate (BOP, Castro's Reagent), hydroxybenzatriazole and diisopropylethylamine.

The carboxy protecting group Z is preferably selected from those protecting groups that can be removed to furnish the free carboxylic acid group without cleaving the N—Y bond. Preferably, Z is selected from those protecting groups that can be removed to furnish the free carboxylic acid group without deprotection of the protected amino acid side chains A, where present.

The carboxy protecting group Z is preferably a silyl group. More preferably, it is a group of formula —Si(R²R³R⁴), wherein R², R³ and R⁴ are independently selected from C₁₋₁₀ hydrocarbyl. Still more preferably, R², R³ and R⁴ are independently selected from C₁₋₁₀ alkoxy and C₁₋₁₀ alkyl. Still more preferably, Z is selected from tri-t-butoxysilyl, di-t-butoxymethylsilyl, di-t-butoxyethylsilyl and tri-t-isopropyloxysilyl. Most preferably, Z is tri-t-butoxysilyl.

The linker group Y is a chemical bond, or chemical moiety capable of forming a covalent bond to both the solid support W and the amine group of an amino acid. Many suitable linker groups are known. Preferably, the Y—N bond of compound (I) above is cleavable to yield the free peptide (VII). Preferred linker groups Y are formylmethyl —CH2—O—(C═O)— and 2-aminoformyl-9-fluorenylmethyl (Fmoc-C═O).

By “capping agent” it is meant a reagent or combination of reagents that is capable of reacting with a free carboxylic acid group to give a derivative. It is highly preferred that the derivative is stable to the conditions employed in step (d) above. By “stable” it is meant that the derivative undergoes less than 20% reversion to the free carboxylic acid during step (d). Preferably, the derivative undergoes less than 10% reversion to the free carboxylic acid during step (d). More preferably, the derivative undergoes less than 1% reversion to the free carboxylic acid during step (d).

Preferably, the capping agent is capable of reacting with a free carboxylic acid group to give an ester, amide or thioester.

In a preferred embodiment, the capping agent is capable of reacting with a free carboxylic acid group to give an ester. Preferably, the capping agent is capable of reacting with a free carboxylic acid group to give an alkyl ester, more preferably a C₁₋₆ alkyl ester. Most preferably, the capping agent is capable of reacting with a free carboxylic acid to give a methyl ester.

Preferred capping agents are alkylating agents. In this context, an alkylating agent is a reagent or combination of reagents capable of reacting with a free carboxylic acid to give an alkyl ester. Suitable alkylating agents include alkyl halides, sulphates, sulphonates, and diazoalkyl compounds.

In a particularly preferred embodiment, the capping reagent comprises diazomethane and/or a diazomethane equivalent such as trimethylsilyl diazomethane.

After every activation and coupling step (steps (a) and (b) above) there remains a proportion of starting material (II) which has not reacted. The capping step (c) ensures that the free carboxylic acid group of this impurity is converted to a stable derivative, and is thus unable to participate in further amino acid couplings. The presence of deletion artefacts is thus greatly reduced.

In a very highly preferred embodiment, the protecting group Z employed in the process is tri-t-butoxysilyl, and the capping agent comprises or is diazomethane. Using this combination of protecting group and capping agent it has been found that free carboxylic acid contaminants (II) are rapidly and almost quantitatively converted to the corresponding methyl ester. The methyl esters thus formed are highly resistant to the conditions used in the deprotection step (d) above.

The skilled person will readily appreciate that steps (a) and (b) can be carried out in one operational step; for instance, the solid support-bound amino acid derivative (II) may be activated in the presence of carboxy-protected amino acid or peptide derivative (IV), without the necessity of isolating activated form (III).

The skilled person will moreover appreciate that before and after each step (a), (b), (c) and (d) it may be necessary to swell the resin with a suitable solvent to enable the reagent to permeate fully and react completely with the bound peptide. Furthermore, after each step (a), (b), (c) and (d) it may be necessary or expedient to wash the resin to remove excess reagent, byproducts and impurities.

Steps (a) and (b) may be repeated if a significant proportion of carboxylic acid (II) remains unreacted (“recoupling”).

Suitable conditions for effecting steps (a) and (b) in terms of temperature, solvent and duration of reaction may be ascertained by routine experimentation.

Suitable conditions for the removal of the protecting group Z in step (d) above will depend on the nature of that group, and will be apparent to one skilled in the art. When Z is tri-t-butyloxysilyl, step (d) may be conducted with mild acid or mild base. Preferred conditions for step (d) wherein Z is tri-t-butyloxysilyl are treatment with dilute trifluoroacetic acid in the presence of organic solvent. More preferred conditions for step (d) wherein Z is tri-t-butyloxysilyl are treatment with 25% trifluoroacetic acid in dichloromethane.

Optionally, the solid support-bound peptide (I) may be cleaved from the solid support to give a free peptide (VII). The cleavage conditions will depend on the nature of the group Y. When the solid support is a modified Merrifield resin, and the linker group Y is —CH₂O(C═O)—, the peptide may be cleaved from the solid support by treatment with HF or trifluoromethanesulfonic acid (TFMSA). Cleavage may occur with concomitant removal of side-chain protecting groups (where present).

In a preferred embodiment, the free peptide (VII) is liberated from the solid-support bound carboxy-protected peptide (X) in one step. This may be achieved for example with HF or trifluoromethanesulfonic acid (TFMSA).

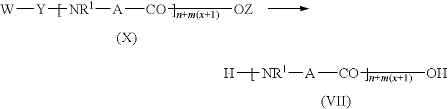

In one embodiment, the solid-support bound carboxy-protected peptide (X) may be cleaved from the solid support to give a carboxy-protected peptide (XI).

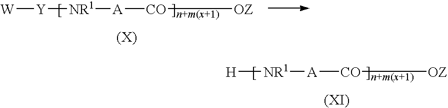

In another preferred embodiment, compound (I) may be subjected to C-terminal modifaction to give a solid support-bound peptide analogue. Examples of C-terminal modifications include esters, thioesters, alcohols, diols and aldehydes.

Preferably, R¹ is hydrogen, hydrocarbyl, or A, taken together with R¹ and N, forms a heterocycle. More preferably, R¹ is hydrogen, C₁₋₆ alkyl, or C₁₋₆ acyl, or A, taken together with R¹ and N, forms a heterocycle

The term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

The present invention will be described in more detail with reference to the following non-limiting examples.

### EXAMPLES

Abbreviations: DCC, N,N′-dicyclohexylcarbodiimide; SPPS, solid-phase peptide synthesis; RP-HPLC, reversed-phase high-performance liquid chromatography; HOBt, 1-hydroxybenzotriazole; BOP, benzotriazole-1-yloxytris(dimethylamino)phosphonium hexafluorophosphate; TLC, thin-layer chromatography; FIB-MS, fast ion bombardment mass spectrometry; ES-MS, electrospray mass spectrometry; MALDI-TOF, matrix assisted laser desorption ionisation-time of flight; DIPEA, diisopropylethylamine; DMAP, N,N-′dimethylaminopyridine; Boc, tert-butyloxycarbonyl; Fmoc, 9-fluorenylmethoxycarbonyl; DCM, dichloromethane; TFMSA, trifluoromethane sulfonic acid; DMF, N,N-dimethylformamide; HVTLE, high voltage thin-layer electrophoresis; Tbos, tri-tert-butoxysilyl; TFA, trifluoroacetic acid; HF, hydrogen fluoride; PEG2000-OH, Polyethylene glycol 2000 monomethyl ether; HCTU, O-(1H-6-Chlorobenzotriazole-1-yl)-1,1,3,3-tetramethyluronium hexafluorphosphate; Other abbreviations correspond to standard nomenclature used for naturally occurring amino acids. All amino acids except glycine are of the I-configuration unless otherwise specified. [Standard abbreviations for amino acids, peptides and protecting groups follow the recommendations of the IUPAC-IUB Joint Commission on Biochemical Nomenclature (1984) Eur. J. Biochem. 138, 9-37.]

2. General Materials and Methods

Unless otherwise stated, all solvents and reagents obtained from various commercial sources were of highest grade and were used without further purification. DMF was stored over molecular sieve 4 Å. Proton nuclear magnetic resonance spectra were recorded on a Hitachi Perkin Elmer R1500 60 MHz instrument in CDCl₃ and chemical shifts are reported as δ units (ppm) relative to tetramethyl silane. TLC was performed on Merck 60 F₂₅₄ precoated silica gel plates using solvent systems (v/v): (A) CHCl₃—MeOH, 9:1 and (B) diethylether-light petroleum (60-80° C.), 7:3. Reaction products were visualised by UV fluorescence (254 nm), 2% ninhydrin in ethanol or by using iodine vapour. Column chromatography was carried out on Merck (230-400 mesh) silica gel. Optical rotations were measured on a Perkin Elmer 141 polarimeter (sodium lamp, 589 nm) at 21° C. Analytical and preparative reversed-phase HPLC (RP-HPLC) experiments were performed on a Gilson 715 instrument equipped with a multi-wave length detector (Applied Biosystems 759A) and two slave 306 pumps. Retention times are given for gradient elution using the following conditions: Column, Vydac C₁₈ (10 m, 0.46 and 2.2×25 cm); eluant A, 0.1% (v/v) TFA in H₂O; eluant B, 0.1% (v/v) TFA in acetonitrile; gradient, 0% over 2 min., 0-80% over 32 min., flow rate, 1 ml/min (analytical) and 10 ml/min (preparative); absorbance, 216 and 235 nm. Molecular weight determinations were carried out by fast ion bombardment (FIB), on a TS250 VG, matrix assisted laser desorption ionisation-time of flight (MALDI-TOF), Perceptives Biosystems Voyager and electrospray (ES) Micromass Quattro 11 mass spectrometers. Infrared spectra were recorded as thin film or in Nujol mull on a Pye Unicam SP3-200 instrument. The accurate mass determination of TBos amino acid esters were performed using a direct probe (EI) approach with suitable internal standards.

3. Preparation of Amino Acid Tri-tert-butoxysilyl Esters (TBos Esters)

General Procedure

An amino acid (50 mmol) was suspended in tert-butanol (40 ml) containing anhydrous pyridine (16.8 ml, 210 mmol) in a 250 ml two-necked flask fitted with a calcium chloride drying tube and a rubber septum. The mixture was cooled to 0° C. (ice bath) and stirred. Silicon tetrachloride (5.73 ml, 50 mmol) was added drop wise with care and was stirred for two hours at room temperature and then at 50° C. for a further 30 minutes. The mixture was allowed to cool; the precipitated pyridinium hydrochloride filtered through a bed of Celite and was washed with ethyl acetate (50 ml). The solvents were removed under reduced pressure (rotary evaporation) and the resultant oily residue dissolved in ethyl acetate (50 ml), transferred to a separating funnel and was washed with H₃PO₄ solution (1M, 10 ml), water (10 ml), NaHCO3 (10% w/v, 20 ml), brine (2×10 ml), and then dried (Na SO4). Removal of the solvents under reduced pressure gave the target compound as a syrup which was used without further purification for peptide synthesis or stored under argon at 4° C. until further use. On prolonged storage, TBos esters have been found to give a white precipitate of a polymeric form of silicon oxide, which can easily be removed by dissolving the ester in acetonitrile followed, by filtration and removal of solvent under reduced pressure. All TBos esters were purified by flash column chromatography using silica gel, which was pre-washed with 1% triethylamine in chloroform. The required ester was obtained by eluting with chloroform-methanol (95:5,v/v). The TBos esters of all naturally occurring amino acids were synthesised in this manner and gave satisfactory HPLC and TLC analysis.

Alanine-tri-tert-butoxysilyl ester. Oil; Yield 11.0 g, 66%; Rf_{A}=0.69; ESMS, m/z 336 [M+H]⁺; IR (thin film) 3450-3320 (br, NH₂), 1750 (s, C═O str, ester), 1260, 1195 (s, C—O), 1110 (s, Si—O) cm⁻¹; ¹H-NMR (60 MHz, CDCl₃) δ 1.14 (s, 27 H, Si[OC(CH₃)₃]₃), 1.6 to 1.8 (d, 3 H, CH₃) and 3.85 to 4.15 (m, 1 H, α-proton).

Arginine-(N^{G}—NO₂)-tri-tert-butoxysilyl ester. Oil; Yield 12.4 g, 53%; Rf_{A}=0.34; ESMS, m/z 466 [M+H]⁺; IR (thin film) 3500-3200 (br, NH₂), 1750 (s, C═O str, ester), 1260, 1100 (s, C—O), 970 (s, Si—O) cm⁻¹.

Asparagine-tri-tert-butoxysilyl ester. Oil; Yield 14.0 g, 74%; Rf_{A}=0.59; ESMS, m/z 379 [M+H]⁺; IR (thin film) 3500-3300 (br, NH₂), 1750 (s, C═O str, ester), 1265, 1100 (s, C—O), 970 (s, Si—O) cm⁻¹.

Aspartate-(OBzl)-tri-tert-butoxysilyl ester. Oil; Yield 19.7 g, 83%; Rf_{A}=0.71; ESMS, m/z 470 [M+H]⁺; IR (thin film) 3450-3320 (br, NH₂), 1750 (s, C═O str, ester), 1260, 1195 (s, C—O), 1110 (s, Si—O) cm⁻¹.

Cysteine-(MeOBzl)-tri-tert-butoxysilyl ester. Oil; Yield 19.8 g, 81%; Rf_{A}=0.68; ESMS, m/z 488 [M+H]⁺; IR (thin film) 3450-3300 (br, NH₂), 1740 (s, C═O str, ester), 1250, 1180 (s, C—O), 1100 (s, Si—O) cm⁻¹; ¹H-NMR (60 MHz, CDCl₃) δ1.29 (s, 27 H, Si[OC(CH₃)₃]₃), 3.05 to 3.2 (m, 2 H, CH₂S), 3.76 (s, 2 H, benzylic-CH2), 3.85 (s, 3 H, OCH₃), 4.15 (m, 1 H, α-proton), 6.60 to 6.85 (d, 2 H, aromatics) and 7.2 to 7.4 (d, 2 H, aromatics).

Glutamate-(OBzl)-tri-tert-butoxysilyl ester. Oil; Yield 19.6 g, 81%; Rf_{A}=0.84; ESMS, m/z 484 [M+H]⁺; IR (thin film) 3420-3200 (br, NH₂), 1750, 1730 (s, C═O str, esters), 1265, 1170 (s, C—O), 1110 (s, Si—O) cm⁻¹; ¹H-NMR (60 MHz, CDCl₃) δ1.27 (s, 27 H, Si[OC(CH₃)₃]₃), 1.82 to 2.75 (m, 4 H, CH₂CH₂), 3.80 to 4.5 (m, 3 H, benzylic-CH₂, α-proton) and 7.2 to 7.5 (m, 5 H, aromatics).

Glutamine-tri-tert-butoxysilyl ester. Oil; Yield 8.2 g, 42%; Rf_{A}=0.40; ESMS, m/z 393 [M+H]⁺; IR (thin film) 3500-3200 (br, NH₂), 1730 (s, C═O str, ester), 1220, 1190 (s, C—O), 1090 (s, Si—O) cm⁻¹.

Glycine-tri-tert-butoxysilyl ester. Oil; Yield 9.89 g, 61%; Rf_{A}=0.68; ESMS, m/z 322 [M+H]⁺; IR (thin film) 3500-3320 (br, NH₂), 1760 (s, C═O str, ester), 1250, 1195 (s, C—O), 1090 (s, Si—O) cm⁻¹; ¹H-NMR (60 MHz, CDCl₃) δ1.32 (s, 27 H, Si[OC(CH₃)₃]₃) and 3.80 to 4.10 (m, 2 H, α-protons).

Histidine-(N^{im}-DNP)-tri-tert-butoxysilyl ester. This derivative was prepared as described in the general procedure except that anhydrous pyridine (20.8 ml, 260 mmol) was used and gave yellow oil. Yield 10.2 g, 36%; Rf_{A}=0.60; ESMS, m/z 568 [M+H]⁺; IR (thin film) 3450-3220 (br, NH₂), 1750 (s, C═O str, ester), 1600, 1530, 1350 (imidazole, DNP), 1260, 1190 (s, C—O), 1080 (s, Si—O) cm⁻¹.

Histidine-(N^{im}-Tosyl)-tri-tert-butoxysilyl ester. Anhydrous pyridine (20.8 ml, 260 mmol) was used in this preparation and gave an oil. Yield 12.9 g, 48%; Rf_{A}=0.58; ESMS, m/z 556 [M+H]⁺; IR (thin film) 3500-3300 (br, NH₂), 1740 (s, C═O str, ester), 1600 (imidazole), 1260, 1200 (s, C—O), 1100 (s, Si—O) cm⁻¹.

Isoleucine-tri-tert-butoxysilyl ester. Oil; Yield 15.1 g, 80%; Rf_{A}=0.67; ESMS, m/z 378 [M+H]⁺; IR (thin film) 3450-3300 (br, NH₂), 1750 (s, C═O str, ester), 1250, 1190 (s, C—O), 1090 (s, Si—O) cm⁻¹.

Leucine-tri-tert-butoxysilyl ester. Oil; Yield 13.8 g, 73%; Rf_{A}=0.71; ESMS, m/z 378 [M+H]⁺; IR (thin film) 3350-3200, 3000, 1750, 1265, 1155 and 1100 cm⁻¹. ¹H-NMR (60 MHz, CDCl₃) δ 0.9-1 (d, 7H), 1.27 (s, 27 H), 1.35-1.74 (m, 2H), and 4.0-4.40 (m, 1 H).

Lysine-(Z)-tri-tert-butoxysilyl ester. Oil; Yield 23.0 g, 87%; Rf_{A}=0.67; ESMS, m/z 527 [M+H]⁺; IR (thin film) 3500 (br, NH₂), 1755, 1720 (s, C═O str, ester, carbamate), 1265 (s, C—O), 1100 (s, Si—O) cm⁻¹.

Lysine (Fmoc)-tri-tert-butoxysilyl ester. Oil; Yield 18.7 g, 61%; Rf_{A}=0.71; ESMS, m/z 615 [M+H]⁺; IR (thin film) 3380 (br, NH₂), 1755, 1710 (s, C═O str, ester, Fmoc), 1265 (s, C—O), 1100 (s, Si—O) cm⁻¹.

Methionine-tri-tert-butoxysilyl ester. Waxy solid; Yield 13.8 g, 70%; Rf_{A}=0.55; ESMS, m/z 395 [M+H]⁺; IR (thin film) 3500-3400 (br, NH₂), 1730 (s, C═O str, ester), 1230 (s, C—O), 1080 (s, Si—O) cm⁻¹; ¹H-NMR (60 MHz, CDCl₃) δ1.27 (s, 27 H, Si [OC (C—H₃)₃]₃), 2.12 (s, 3 H, SCH₃), 2.4 to 2.6 (d, 2 H, CH₂S) and 4.2 to 4.64 (m, 1 H, α-proton).

Phenylalanine-tri-tert-butoxysilyl ester. Oil; Yield 15.7 g, 76%; Rf_{A}=0.73; ESMS, m/z 411 [M+H]⁺; IR (thin film) 3300-3100 (br, NH₂), 1745 (s, C═O str, ester), 1265 (s, C—O), 1100 (s, Si—O) cm⁻¹; ¹H-NMR (60 MHz, CDCl₃) 1.27 (s, 27 H, Si[OC(CH₃)₃]₃), 3.0 to 3.4 (d, 2 H, benzylic-CH₂), 4.25 to 4.5 (m, 1 H, α-proton) and 7.25 (m, 5 H, aromatics).

Proline-tri-tert-butoxysilyl ester. Oil; Yield 13.5 g, 75%; Rf_{A}=0.80; ESMS, m/z 361 [M+H]⁺; IR (thin film) 1745 (s, C═O str, ester), 1265 (s, C—O), 1110 (s, Si—O) cm⁻¹; ¹H-NMR (60 MHz, CDCl₃) δ1.26 (s, 27 H, Si [OC (CH₃)₃]₃), 1.7 to 2.45 (m, 4 H, CH₂CH₂), 3.35 to 3.75 (m, 2 H, CH₂ and 4.2 to 4.5 (m, 1 H, α-proton).

Serine-(Bzl)-tri-tert-butoxysilyl ester. Oil; Yield 14.4 g, 65%; Rf_{A}=0.75; ESMS, m/z 441 [M+H]⁺; IR (thin film) 3500-3320 (br, NH₂), 1750 (s, C═O str, ester), 1260, 1195 (s, C—O), 1110 (s, Si—O) cm⁻¹; ¹H-NMR (60 MHz, CDCl₃) δ1.27 (s, 27 H, Si[OC(CH₃)₃]₃), 3.6 to 3.7 (d, 2 H, CH₂O), 4.0 to 4.3 (m, 3 H, benzylic-CH₂, α-proton) and 7.28 (s, 5 H, aromatics).

Threonine-(Bzl)-tri-tert-butoxysilyl ester. Oil; Yield 16.6 g, 73%; Rf_{A}=0.77; ESMS, 455 [M+H]⁺; IR (thin film) 3450-3320 (br, NH₂), 1750 (s, C═O str, ester), 1250, 1195 (s, C—O), 1110 (s, Si—O) cm⁻¹; ¹H-NMR (60 MHz, CDCl₃) δ1.2 to 1.8 (m, 30 H, Si[OC(CH₃)₃]₃, CH₃), 4.0 to 4.55 (m, 3 H, benzylic-CH₂, α-proton) and 7,29 (br.s, 5H).

Tryptophan-(formyl)-tri-tert-butoxysilyl ester. Oil; Yield 13.1 g, 51%; Rf_{A}=0.81; ESMS, m/z 479 [M+H]⁺; IR (thin film) 3500-3400 (br, NH₂), 1760, 1680 (s, C═O str, ester, formyl), 1200 (s, C—O), 1100 (s, Si—O) cm⁻¹.

Tryptophan-tri-tert-butoxysilyl ester. Oil; Yield 16.2 g, 72%; Rf_{A}=0.58; ESMS, m/z 452 [M+H]⁺; IR (thin film) 3500-3300 (br, NH₂), 1750 (s, C═O str, ester), 1200 (s, C—O), 1110 (s, Si—O) cm⁻¹.

Tyrosine-(Bzl)-tri-tert-butoxysilyl ester. Oil; Yield 20.2 g, 78%; Rf_{A}=0.90; ESMS, m/z 518 [M+H]⁺; IR (Thin film) 1750 (s, C═O str, ester), 1260, 1195 (s, C—O), 1110 (s, Si—O) cm⁻¹.

Valine-tri-tert-butoxysilyl ester. Oil; Yield 14.1 g, 78%; Rf_{A}=0.62; ESMS, m/z 363 [M+H]⁺; IR (thin film) 3500-3320 (br, NH₂), 1750 (s, C═O str, ester), 1250, 1195 (s, C—O), 1110 (s, Si—O) cm⁻¹; ¹H-NMR (60 MHz, CDCl₃) δ1.05 to 1.3 (m, 6 H, C[CH₃]₂), 1.31 (s, 27 H, Si[OC(CH₃)₃]₃), 2.15 to 2.40 (m, 1 H, CH) and 3.85 to 4.10 (m, 1 H, α-proton).

4. Preparation of Resin

Derivatisation of tri-tert-butoxysilylamino acid ester with Merrifield chloromethyl resin was achieved in accordance with the methods described by Merrifield and others (Letsinger, R. L. and Kornet, M. J. (1963) J. Amer. Chem. Soc., 85, 3045-3046, Letsinger, R. L., Kornet, M. J., Mahadevan, V. and Jerina, D. M. (1964) J. Amer. Chem. Soc., 86, 5163, Felix, A. M. and Merrifield, R. B. (1970) J. Amer. Chem. Soc., 92, 1385-1391).

5 Formation of Methylchloroformylated Resin

Chloromethylated co-polystyrene-2% divinylbenzene (5 g, 1 mmol/g) was suspended in 2-methoxyethanol (40 ml) and was treated with potassium acetate (1.4 g, 14.2 mmol) at 130° C. for 72 hours. The reaction mixture was allowed to cool to room temperature and filtered. The resin was washed with water (100 ml), methanol (100 ml), diethyl ether (100 ml), respectively, and dried (Infrared spectra, 1740 cm⁻¹). It was treated with NaOH (0.5M, 40 ml) at room temperature for 72 hours to complete the reaction. The resin was then washed with water, methanol, diethyl ether as before and dried under vacuo. The Infrared spectrum showed the absence of 1740 cm⁻¹ band. The hydroxymethyl resin was then treated with phosgene (20% solution in toluene, 40 ml, 80 mmol) at room temperature for 4 hours. The resin was filtered, washed thoroughly with diethyl ether and dried (Infrared, 1785 cm⁻¹).

6 Attachment of the First Amino Acid to the Solid Support

Attachment of the first amino acid via its amino function was successfully achieved through the benzyloxycarbonyl linkage to Merrifield resin, by the method described by Felix and Merrifield 1970 thus giving a peptide resin linkage that is stable enough to all reagents used during peptide synthesis, and is cleavable by the treatment of strong acid such as HF, HBr or TFMSA.

The resins substitutions were estimated by HBr cleavage (described below), and by the back-titration method after removal of the TBos ester (table 1). The general increase in substitution levels obtained for this work as compared to Jones was attributed to a higher substitution of the hydroxy moiety on the hydroxy methyl Merrifield resin.

7 Estimation of the Level of Substitution of Amino Acid to the Resin by HBr Cleavage

After removal of the TBos ester, the resin was thoroughly washed with DCM (3×10 ml), Et₂O (2×10 ml) and dried under vacuum. Approximately 5 mg of resin was accurately weighed and placed in a clean micro-centrifuge tube. HBr in glacial acetic acid (0.2 ml, 30% wt/v) was added, the tube sealed and agitated by rotating mixer (10 rpm) for 4 hr at room temperature. The tube was carefully pierced in the cap and evacuated to dryness. The residue was diluted with MeOH (1 ml), filtered to remove the resin, washed with more MeOH (1 ml), and the filtrates pooled. An aliquot of this solution (50 μl) was subjected to a quantitative ninhydrin assay.

TABLE 1
The estimated substitution of the benzyloxycarbonyl TBos ester
resins.

| Benzyloxycarbonyl TBos | Estimated Substitution |
|---|---|
| ester resins | (mmol/g) |
| Alanine | 0.47 |
| Asparagine | 0.40 |
| Arginine (NO₂) | 0.45^{†} |
| Cysteine (MeOBzl) | 0.46 |
| Glycine | 0.35 |
| Isoleucine | 0.50 |
| Leucine | 0.50 |
| Lysine (NαFmoc) | 0.44 |
| Lysine (Z) | 0.30^{‡} |
| Methionine | 0.45 |
| Phenylalanine | 0.32 |
| Proline | 0.31 |
| Threonine (Bzl) | 0.35 |
| Tyrosine (Bzl) | 0.18 |
| Valine | 0.41 |

7 Peptide Synthesis

Having ascertained the optimal reaction conditions for this form of peptide synthesis, attention was focussed upon obtaining a longer peptide. This was a 5-mer Leucine enkephalin. This peptides was chosen because some of the peptides are already well characterised by biological activity assays and by synthesis by conventional means in our laboratory, and therefore serve as a direct comparison. To verify that the synthesises (monitored by back-titration unless otherwise stated) were proceeding according to their schedules, at intervals, some of the peptidyl-resin was cleaved by high HF or HBr and the product analysed by RP-HPLC, and mass spectrometry techniques.

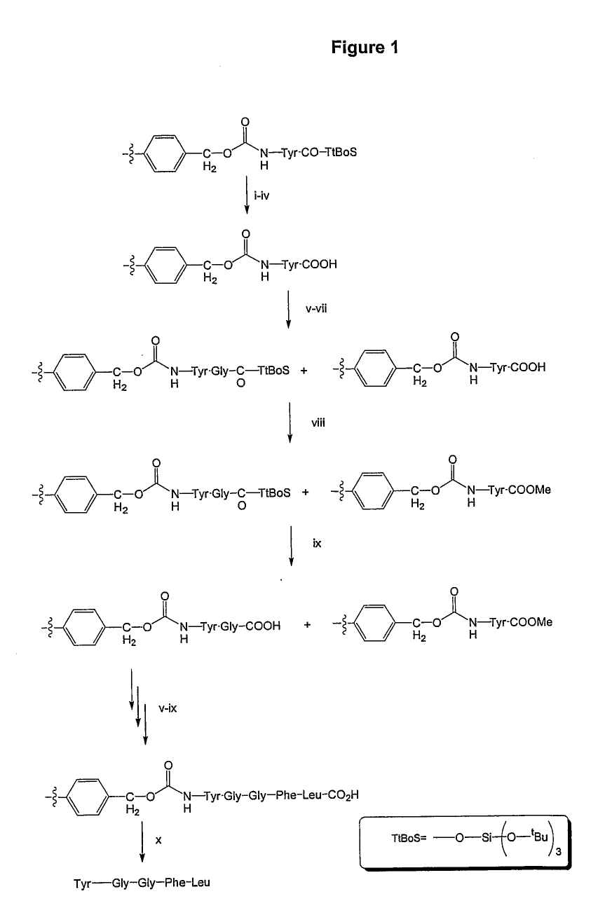

All peptides were assembled manually on the solid phase from the N to C direction (a reaction protocol for N to C synthesis is illustrated in FIG. 1 upon a 0.1 mmol scale).

FIG. 1 shows the synthesis of leucine-enkephalin on the solid phase in the N→C direction. The solvent was 6 mL throughout for 0.5 g of resin; reagents and conditions: i, wash, CH₂Cl₂ (2×1 min); ii, deprotect, 25% TFA-CH₂Cl₂ (2×5 min); iii, wash, CH₂Cl₂ (3×1 min), DMF (1 min); iv (OPTIONAL), monitoring, remove 3-5 mg resin for assay; v, coupling, T-t-Bos amino acid (4 fold excess): BOP:HOBt:DIPEA (1:1:1:2 equiv), DMF, 60 min; vi, wash, DMF (2×1 min); vii, repeat iv; viii, CH₂N₂ ix, repeat ii and iii; x, cleavage, HF or TFMSA; xi, purification, RP-HPLC. When necessary the amino acid derivatives were recoupled. Coupling and recoupling times were never longer than one hour.

The TBos group was used for temporary carboxyl protection of amino acids and side chain protecting groups were: Tyr (Bzl), Thr (Bzl), Lys (Z), Glu (OBzl), Ser (Bzl), Cys (MeOBzl), Trp (Formyl), and Arg (NO₂). All couplings were performed in DCM and whenever necessary, a second coupling was carried out. The peptides were cleaved by high HF, and purified by standard RP-HPLC.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. A process for the preparation of a solid support-bound peptide of formula (I) (I) 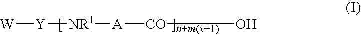
which comprises the following steps:
(a) reacting a solid-support bound amino acid or peptide derivative (II) with a carboxyl group activating agent to form an activated solid support-bound compound of formula (III): 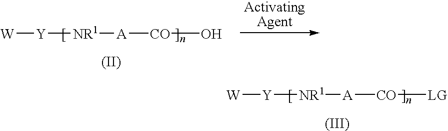
(b) reacting the activated solid support-bound compound of formula (III) with a second carboxy-protected amino acid or peptide derivative of formula (IV) to form a solid support-bound peptide chain extended compound of formula (V); 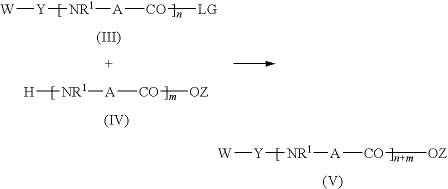
(c) treating the solid support-bound peptide chain extended compound of formula (V) with a capping agent;
(d) removing the protecting group Z to produce a solid support-bound peptide derivative of formula (VI); 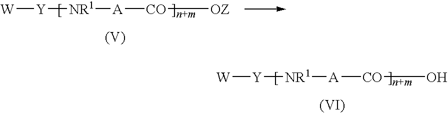
(e) repeating steps (a), (b), (c) and (d) x times to form the desired compound of general formula (I); 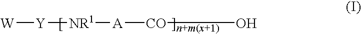
wherein
n is a positive integer
m is a positive integer
x is O or a positive integer
Z is a carboxy protecting group
W is a solid support
Y is a linker group or a chemical bond
LG is a leaving group
R¹ is hydrogen or a substituent
and for each A, which may be the same or different,
i) A represents the amino acid residue; or
ii) A, taken together with R1 and N, forms a heterocycle.

2. A process according to claim 1 wherein the solid support-bound peptide chain extended compound of formula (V) comprises as an impurity an amount of solid support-bound free carboxylic acid or a salt form thereof.

3. A process according to claim 1 or 2 wherein the capping agent is a reagent or combination of reagents that is capable of reacting with a free carboxylic acid or a salt form thereof to form a derivative.

4. A process according to claim 3 wherein the derivative is stable to the conditions of step (d) as defined in claim 1.

5. A process according to claim 3 wherein the derivative is an ester.

6. A process according to claim 5 wherein the derivative is an alkyl ester.

7. A process according to claim 5 wherein the derivative is a methyl ester.

8. A process according to claim 1 wherein the capping agent is a diazoalkyl compound.

9. A process according to claim 1 wherein the capping agent is diazomethane or a diazomethane equivalent.

10. A process according to claim 1 wherein the protecting group Z is a group of formula —Si(R²R³R⁴), wherein R², R³ and R⁴ are independently selected from C₁₋₁₀ hydrocarbyl.

11. A process according to claim 10 wherein the protecting group Z is a tri-f-butoxysilyl group.

12. A process according to claim 1 wherein R¹ is hydrogen, C₁₋₆ alkyl, or C₁₋₆ acyl, or A, taken together with R¹ and N, forms a heterocycle.

13. A process according to claim 1 comprising a further step of cleaving a solid support-bound peptide of general formula (I) from the solid support to give a free peptide of formula (IX) 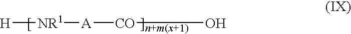
or a salt form thereof.

14. A process for solid-phase N—C peptide synthesis comprising a step of treating a mixture of:
a C-terminal protected amino acid sequence including one or more amino acids obtainable by N—C synthesis linked to a resin and;
an impurity comprising a free carboxylic acid group or a salt form thereof linked to a resin;
with an alkylating agent.

15. A process according to claim 14 wherein the alkylating agent is diazomethane.

16. A solid support-bound peptide obtained by the process of any one of claims 1 or 14.

17. A peptide obtained by the process of claim 13.

18. (canceled)

19. (canceled)

20. (canceled)
